(19) United States
(12) Patent Application Publication
(10) Pub. No.: US 2025/0359932 A1
(43) Pub. Date: Nov. 27, 2025

(54) POWER DENSITY BOOSTING FIBER FOR MEDICAL LASER SYSTEMS

(71) Applicant: LUMENIS LTD., Yokneam Illit (IL)

(72) Inventors: Arkady Khachaturov, Kiryat Yam (IL); Tal Waisman, Haifa (IL)

(73) Assignee: LUMENIS LTD., Yokneam Illit (IL)

(21) Appl. No.: 19/204,922

(22) Filed: May 12, 2025

Related U.S. Application Data
(60) Provisional application No. 63/650,488, filed on May 22, 2024.

(51) Int. Cl. A61B 18/24; A61B 1/018; A61B 17/00; A61B 18/00; A61B 18/22; A61B 90/98

(52) U.S. Cl. CPC A61B 18/24; A61B 1/018; A61B 90/98; A61B 2017/00154; A61B 2017/00199; A61B 2018/00172; A61B 2018/00702; A61B 2018/2244

(57) ABSTRACT

The present disclosure provides medical optical fibers configured to couple to medical laser systems and convey laser energy to a target. The medical optical fibers comprise a fiber core surrounded by a cladding which is further surrounded by a jacket. The overall diameter of the jacket is configured for use with conventional endoscopes, but the diameter of the fiber core is configured to convey the laser energy at power densities to cause interactions between the laser energy and the target and between the laser energy and the liquid medium in which the target is found.

### CROSS-REFERENCE TO RELATED APPLICATIONS

This application claims the benefit of U.S. Provisional Application No. 63/650,488, filed May 22, 2024, the disclosure of which is incorporated herein by reference in its entirety.

### TECHNICAL FIELD

The present disclosure generally relates to medical laser systems and particularly, but not exclusively, the present disclosure relates to medical optical fiber coupled to a medical laser system.

### BACKGROUND

Medical lasers are used in a variety of procedures. Among several of the procedures, laser energy is directed towards a target using a fiber as a conduit for the laser energy. For example, ureteral endoscopy, or lithotripsy uses laser energy to address renal calculi (e.g., kidney stones). As another example, laser energy can be applied to address soft tissue abnormalities, such as benign prostatic hyperplasia (BPH). A typical treatment for BPH uses laser energy to enucleate the prostate tissue.

In an example lithotripsy procedure, an endoscopic probe, with a camera or other sensor, is inserted into the patient's urinary tract to locate the calculi for removal. An optical fiber is inserted through the working channel of the endoscope and laser energy is conducted to the calculi to break up, disintegrate, or otherwise irradiate the calculi as they are found.

It is to be appreciated that calculi in such environments are often immersed in a liquid medium and are treated while free floating in the liquid medium. One of the main requirements for lasers systems used in lithotripsy is the ability to convey laser energy to the calculi through the liquid medium. Likewise soft tissue treatments often require precise doses of laser energy.

### BRIEF SUMMARY

The present disclosure provides medical optical fibers configured to couple to medical laser systems and convey laser energy to a target where the laser energy is conveyed at power densities to cause interactions between the laser energy and the target and between the laser energy and the liquid medium in which the target is found.

In particular, the present disclosure provides medical optical fibers with smaller than conventional fiber cores, but which are constructed and include feature that provide rigidity and handling characteristics of medical optical fibers with larger core diameters.

In some embodiments, the invention can be implemented as a medical optical fiber. The medical optical fiber can comprise a proximal connector, a distal tip, and an elongate shaft extending between the proximal connector and the distal tip, the elongate shaft comprising a fiber core; a cladding surrounding the fiber core; and a jacket surrounding the cladding, wherein a ratio of the diameter of the fiber core over the diameter of the elongate shaft is less than or equal to 0.75.

In further embodiments of the medical optical fiber, the ratio of the diameter of the fiber core over the diameter of the elongate shaft less than or equal to 0.5.

In further embodiments of the medical optical fiber, the ratio of a diameter of the cladding over the diameter of the fiber core is greater than or equal to 1.5.

In further embodiments of the medical optical fiber, the ratio of a diameter of the cladding over the diameter of the fiber core is greater than or equal to 2.5

In further embodiments of the medical optical fiber, the ratio of a diameter of the cladding over the diameter of the fiber core is greater than or equal to 1.05 and less than or equal to 1.25.

In further embodiments of the medical optical fiber, the ratio the diameter of the jacket over a diameter of the cladding is greater than or equal is greater than or equal to 1.5.

In further embodiments of the medical optical fiber, the ratio the diameter of the jacket over a diameter of the cladding is greater than or equal is greater than or equal to 2.5.

In further embodiments of the medical optical fiber, the proximal connector is configured to mechanically couple the medical optical fiber to a laser console and to optically couple the fiber core to the laser console.

In further embodiments of the medical optical fiber, the proximal connector comprises radio frequency identification (RFID) circuitry configured to authenticate the medical optical fiber to the laser console.

With some embodiments, the invention can be implemented as a medical laser system. The medical laser system can comprise a laser console comprising a laser source and a controller, the controller configured to identify laser pulse parameters and to cause the laser source to generate laser energy having the laser pulse parameters; and a medical optical fiber according to any one of the examples provided herein.

In further embodiments of the medical laser system, the laser pulse parameters comprise pulse power of 1.5 kilo-Watts (KW).

In further embodiments of the medical laser system, the diameter of the fiber core is such that the medical optical fiber, responsive to receiving laser energy from the laser console having 1.5k W of pulse power delivers laser energy from a distal end of the elongate shaft having a power density of 3.6 Mega-Watts (MW) per centimeter (cm) squared (MW/cm2).

In further embodiments of the medical laser system, the laser pulse parameters comprise pulse power of less than or equal to 2.5 kilo-Watts (kW).

In further embodiments of the medical laser system, the diameter of the fiber core is such that the medical optical fiber, responsive to receiving laser energy from the laser console having less than or equal to 2.5 kW of pulse power delivers laser energy from a distal end of the elongate shaft having a power density of greater than or equal to 3 Mega-Watts (MW) per centimeter (cm) squared (MW/cm2).

In further embodiments, the medical laser system can comprise an endoscope having a working channel configured to receive the medical optical fiber, wherein the working channel comprises an inner diameter (ID) of between 3.2 and 3.8 French (F).

With some embodiments, the disclosure can be implemented as a medical optical fiber. The medical optical fiber can comprise a proximal connector, a distal tip, and an elongate shaft extending between the proximal connector and the distal tip, the elongate shaft comprising: a fiber core; a cladding surrounding the fiber core; and a jacket surrounding the cladding, wherein a diameter of the fiber core is greater than or equal to 100 millimeters (mm) and less than or equal to 200 mm, and wherein a diameter of the elongate shaft is greater than or equal to three (3) times the diameter of the fiber core.

In further embodiments of the medical optical fiber, the ratio of a diameter of the cladding over the diameter of the fiber core is greater than or equal to 2.5.

In further embodiments of the medical optical fiber, the ratio a diameter of the jacket over a diameter of the cladding is greater than or equal is greater than or equal to 2.5.

In further embodiments of the medical optical fiber, the proximal connector is configured to mechanically couple the medical optical fiber to a laser console and to optically couple the fiber core to the laser console.

In further embodiments of the medical optical fiber, the proximal connector comprises radio frequency identification (RFID) circuitry configured to authenticate the medical optical fiber to the laser console

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1 illustrates a medical laser system in accordance with at least one embodiment.

FIG. 2A and FIG. 2B illustrate cross-sectional views of a first medical optical fiber in accordance with at least one embodiment.

FIG. 3A and FIG. 3B illustrate cross-sectional views of a second medical optical fiber in accordance with at least one embodiment.

FIG. 4 illustrates a perspective view of a third medical optical fiber in accordance with at least one embodiment.

FIG. 5 illustrates an endoscope used with medical optical fibers of the present disclosure.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

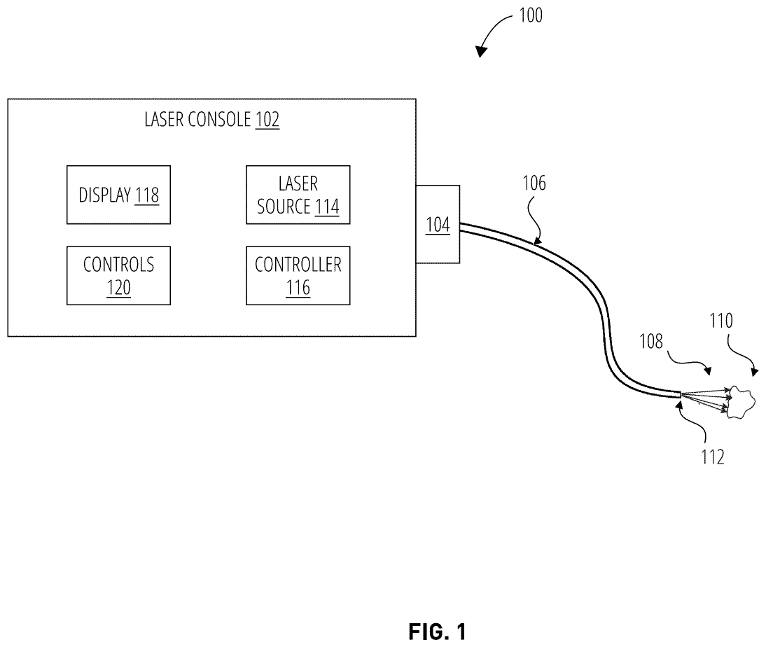

FIG. 1 shows an exemplary medical laser system 100 for generating a pulsed laser beam to treat a target. In general, the medical laser system 100 comprises a laser console 102, optical fiber coupler 104, and optical fiber 106. The optical fiber 106 can be coupled to laser console 102 via the optical fiber coupler 104 for use. During operation, medical laser system 100 can generate laser energy 108 to treat target 110. In general, laser energy 108 can be any laser energy. In some embodiments, laser energy 108 can be a pulsed laser beam. The laser energy 108 can be directed to target 110 by positioning the distal end 112 of the laser energy 108 proximate to the target 110. For example, in some embodiments, target 110 can be a tissue, a stone, a tumor, a cyst, and the like located in a lumen or cavity in a patient's body. As a specific example, target 110 can be a urinary stone located in a kidney or other urinary track, lumen, or cavity. Often, target 110 can be free floating in a liquid medium (e.g., urine, water, etc.) The laser energy 108 can be inserted into the patient's cavity via a working channel of a ureteroscope (not shown) and positioned such that the distal end 112 extends out of the working channel and is proximate to the target 110. The target 110 can be exposed to the laser energy 108 to treat (e.g., ablate, disintegrate, dust, shatter, or the like) the target 110.

Laser console 102 can include laser source 114, controller 116, display 118, and/or controls 120. Laser source 114 can include a laser medium and a pump light source (neither shown) configured to generate laser energy 108. The laser source 114 can include any of a variety of laser mediums and pump light sources. For example, laser source 114 can be a Holmium (Ho) based laser (e.g., Ho: YAG, or the like), a Chromium (Cr), Thulium (Tm), and Ho based laser (e.g., CTH: YAG, or the like). As another example, the laser source 114 can be a Thulium (Tm) based fiber laser (TFL). The pump light sources can be capacitor driven flash lamps, light emitting diodes (LEDs), or the like. Laser source 114 can be optically coupled with optical fiber coupler 104 via any of a variety of optical elements (e.g., lenses, polarizers, beam splitters, beam combiners, light detectors, wavelength division multiplexers, collimators, circulators, etc.) configured to shape and/or reduce spherical aberrations in the laser energy 108 and optical couple the laser energy 108 to the optical fiber 110.

The controller 104 may be associated with and/or communicatively coupled the laser source 114, the display 118, and the controls 120. In general, controller 116 can include processing circuitry (e.g., a processor unit, a microcontroller, or the like) and computer-readable memory storing instructions that when executed by the processing circuitry cause the controller 116 to control (e.g., via sending and receiving control signals and/or information elements) the laser source 114 to cause laser source 114 to generate laser energy 108 having the parameters specified via controls 120. Parameters and/or characteristics of the laser energy 108 and/or the treatment of target 110 can be determined by the controller 116 and displayed on display 118. In some embodiments, the parameters are pulse width, frequency, and pulse power.

As noted, medical laser system 100 can be used for lithotripsy procedures where the target 110 is immersed in a liquid medium. Ho: YAG lasers are widely used for medical laser systems (e.g., medical laser system 100, or the like) provided for lithotripsy procedures. An example, medical laser system 100 where the laser source 114 is a Ho: YAG laser source can provide pulse power of approximately 13 kilo-Watts (kW).

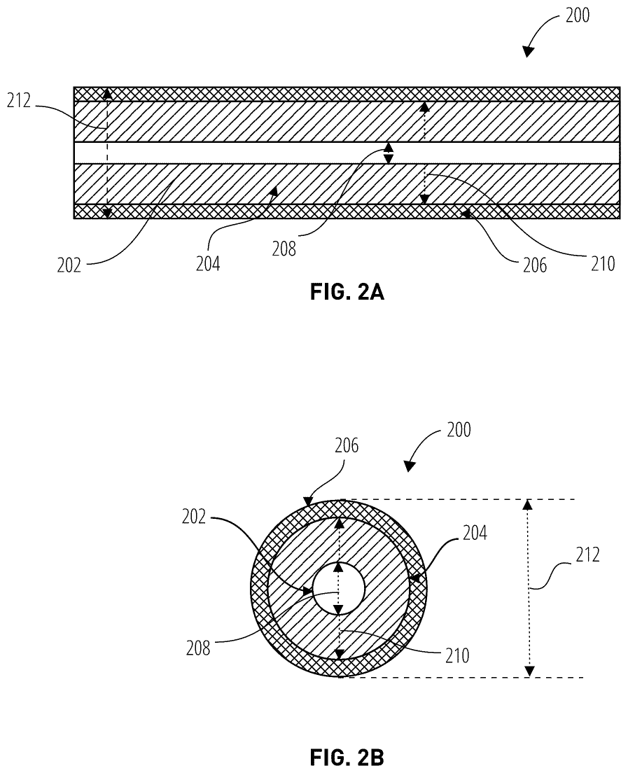

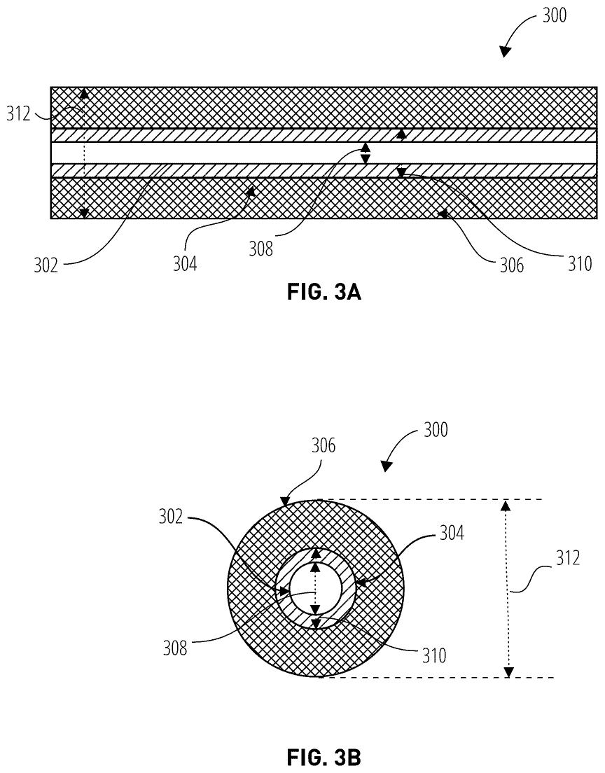

Conventional endoscopes used in lithotripsy procedures, such as a flexible ureteroscope (fURS), often have a working channel diameter (e.g., inner diameter (ID)) of 3 to 4 French (F). For example, the Litho Vue™ Elite fURS has a working channel ID of 3.6F, which is approximately 1.2 millimeters (mm). To that end, optical fiber 106 must have an external diameter of less than or equal to the working channel ID of the fURS. Medical optical fibers (e.g., optical fiber 106, or the like) have a fiber core surrounded by a cladding and jacket (see FIG. 2A, FIG. 2B, FIG. 3A, and FIG. 3B). Accordingly, the outer diameter (OD) of the jacket must be less than the ID of the working channel.

Accordingly, conventional optical fibers 106 used with Ho: YAG laser systems have a fiber core diameter of 0.550 mm. An optical fiber 106 with a fiber core diameter of 0.550 mm when used with a Ho: YAG laser having 13 kW of pulse power provides laser energy with a power density of 5.47 Mega-Watts (MW) per centimeter (cm) squared (MW/cm2), which is sufficient to produce desired clinical effects for lithotripsy procedures.

However, new and/or alternative laser technologies that may be applied to lithotripsy procedures, such as (TFL), have significantly lower pulse power than Ho: YAG lasers. For example, a TFL can be a pulse power of approximately 500W (compared with the 13 kW pulse power of the HO: YAG laser). Accordingly, using conventional optical fibers 106 with TFL laser systems having a fiber core diameter of 0.550 mm results in laser energy having power density of 0.63MW/cm2, which can be insufficient to produce desired clinical results for lithotripsy procedures. Particularly, the power density achievable from TFL laser with conventional optical fibers is insufficient to reliably control the bubble formation during lithotripsy. Furthermore, the reduced power density provides fewer desirable outcomes for soft tissue treatments (e.g., the tissue may undergo charring instead of incision and/or ablation). Likewise, fragmentation and/or dusting of calculi or stones is not as effective as lower power densities.

Accordingly, there is a need for medical optical fibers to be coupled to a medical laser system used for lithotripsy where the medical optical fiber delivers laser energy at power densities sufficient for lithotripsy procedures even where the medical laser system generates laser energy at pulse powers less than conventional systems.

FIG. 2A and FIG. 2B illustrate an optical fiber 200. FIG. 2A illustrates a cross-sectional view of the optical fiber 200 along the longitudinal axis of the optical fiber 200 while FIG. 2B illustrates an on-axis cross-sectional view of the optical fiber 200. With some embodiments, optical fiber 200 can be provided as optical fiber 106 and coupled to medical laser system 100 via optical fiber coupler 104. The optical fiber 200 includes a fiber core 202, a cladding 204 surrounding the fiber core 202 and a jacket 206 surrounding the cladding 204.

With some embodiments, the fiber core 202 (and other fiber cores described herein) can be a fused silica rod configured to convey laser energy (e.g., laser energy 108, or the like). The cladding 204 (and other claddings described herein) can be fused silica doped with various elements (e.g., fluorine, or the like) to alter the refractive index of the cladding 204. The jacket 206 (and other jackets described herein) can be any of a variety of protective coatings, such as, for example, fluoropolymer or the like. In some embodiments, the optical fiber 200 (and other optical fibers described herein) can include a second protective layer disposed between the cladding 204 and jacket 206 (not shown). For example, optical fiber 200 could include a transparent plastic coating over cladding 204 and under jacket 206.

With some embodiments, the fiber core 202 can have a diameter (D) 208 of less than 0.150 mm, less than 0.125 mm, between 0.1 mm and 0.125 mm, or between 0.1 mm and 0.150 mm. In some embodiments the fiber core 202 can have a core D 208 of between 0.100 mm and 0.250 mm, between 100 mm and 200 mm, less than or equal to 250 mm, equal to 0.230 mm, equal to 0.150 mm, equal to 0.120 mm, or equal to 0.100 mm.

In some embodiments, fiber core 202 can have a core D 208 of 0.230 mm. In such an embodiments where optical fiber 106 has a core D 208 of 0.230 mm and where medical laser system 100 generates laser energy having a pulse power of 1.5 kW, laser energy 108 having power density of 3.6MW/cm2 can be emitted from the distal end 112 of the optical fiber 106. For example, optical fiber 200 having the dimensions outlined herein could be provisioned with a TFL medical laser system to produce similar levels of power density as achieved in conventional Ho based laser systems.

It is important to note, that the cladding to core ratio of optical fiber 200 is larger than is typical for conventional optical fibers used with medical laser systems. As used herein, the cladding core ratio is the core D 208 of the cladding 204 over the core D 208 of the fiber core 202. With some embodiments, the cladding to core ratio for the optical fiber 200 can be greater than or equal to 1.5. In some embodiments, the cladding to core ratio for the optical fiber 200 can be greater than or equal to 2. In some embodiments, the cladding to core ratio for the optical fiber 200 can be greater than or equal to 2.5. In some embodiments, the cladding to core ratio for the optical fiber 200 can be between 2 and 3 or equal to 2.85. Accordingly, an optical fiber 200 having a significantly smaller than conventional core D 208 but an overall D 212 that is suitable for use with working channel IDs for conventional fURS can be provided.

It is to be appreciated that the overall D 212 may be necessary for the medical optical fibers to have sufficient fiber rigidity. For example, the medical optical fibers discussed herein are intended to be inserted through a working channel of an endoscope. The present disclosure provides that the overall D 212 can be tuned, regardless of the core D 208, such that the medical optical fiber has sufficient rigidity to pass through the working channel of the endoscope. Further, the overall D 212 can be tuned, regardless of the core D 208, such that that the rigidity is optimized while providing space in the working channel for irrigation fluid flow.

With some embodiments, medical optical fibers, as outlined herein, can be provided with the same core D 208 but with different overall Ds 212, for example, to provide differing volumes of space remaining in the working channel of the endoscope to permit different irrigation fluid flows.

Further still, the overall D 212 of the medical optical fibers provided herein can be tuned, regardless of the core D 208, such that the medical optical fiber has rigidity and handling characteristics suitable for a medical professional (e.g., wearing gloves, operating in a wet environment, or the like) to manipulate the fiber within the working channel of the endoscope.

FIG. 3A and FIG. 3B illustrate an optical fiber 300 with an approximately conventional cladding core ratio but a larger than conventional jacket to core ratio. FIG. 3A illustrates a cross-sectional view of the optical fiber 300 along the longitudinal axis of the optical fiber 300 while FIG. 3B illustrates an on-axis cross-sectional view of the optical fiber 300. With some embodiments, optical fiber 300 can be provided as optical fiber 106 and coupled to medical laser system 100 via optical fiber coupler 104. The optical fiber 300 includes a fiber core 302, a cladding 304 surrounding the fiber core 302 and a jacket 306 surrounding the cladding 304.

In some embodiments the cladding to core ratio for optical fiber 300 can be between 1.1 and 1.3. That is, the cladding D 310 over core D 308 can be between 1.1 and 1.3 while the overall D 312 over cladding D 310 can be significantly larger. For example, in some embodiments, overall D 312 over cladding D 310 can be greater than or equal to 1.5. In some embodiments, the jacket to cladding ratio for the optical fiber 300 can be greater than or equal to 2. In some embodiments, the jacket to cladding ratio for the optical fiber 300 can be greater than or equal to 2.5. In some embodiments, the jacket to cladding ratio for the optical fiber 300 can be between 2 and 3. With some embodiments, the overall D 312 can be greater than or equal to the cladding D 310 plus 0.25 mm. With some embodiments, the overall D 312 can be greater than or equal to the cladding D 310 plus 0.5 mm, With some embodiments, the overall D 312 can be greater than or equal to the cladding D 310 plus between 0.25 mm and 0.5 mm. Accordingly, an optical fiber 300 having a significantly smaller than conventional core D 308 but a conventional cladding to core ratio and an overall D 312 suitable for use with working channel IDs for conventional fURS can be provided.

With some embodiments, the jacket 206 and/or jacket 306 can be made from material having intensive or physical properties (e.g., mechanical bending force, elasticity, fatigue, flexural strength, or the like) to provide optical fiber 200 and/or optical fiber 300 with working characteristics to support the smaller core D 208 and/or core D 308.

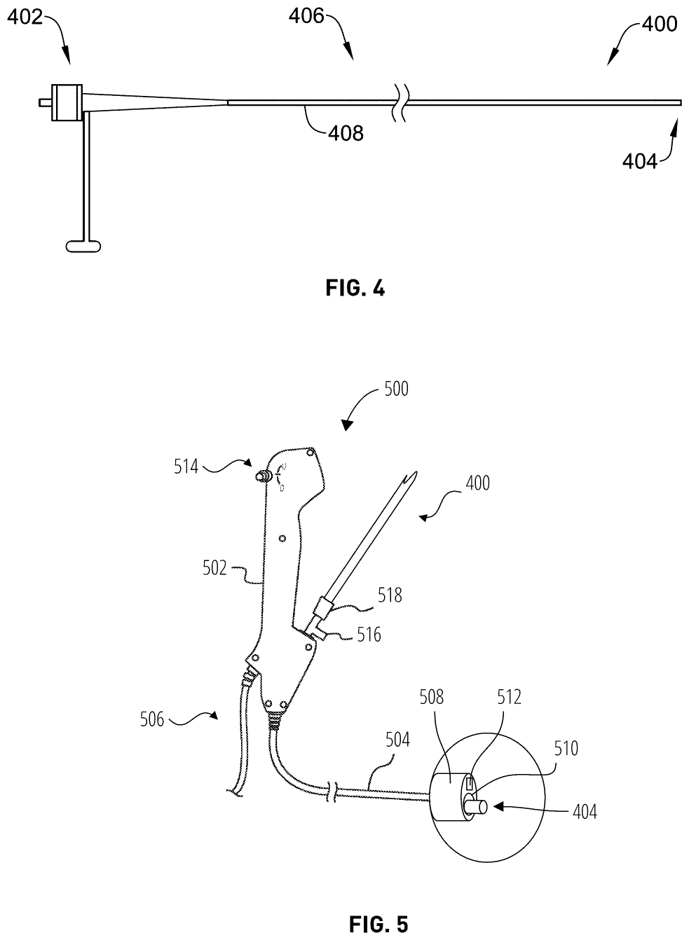

FIG. 4 illustrates an optical fiber 400, which can be provided as the optical fiber 106 of medical laser system 100 to convey laser energy to a target. Optical fiber 400 includes a coupler 402 at its most proximal end and a tip 404 at its most distal end. The coupler 402 can be any of a variety of configurations that facilitate optical coupling between the optical fiber 400 and a laser console, such as, laser console 102. Often, coupler 402 will include mechanical and optical coupling components.

In some embodiments, coupler 402 can include electrical and/or communication components configured to authenticate the optical fiber 400 to the laser console (e.g., radio frequency identification (RFID) circuitry, or the like. The tip 404 can include a variety of features such as a polished facet and/or a protective ball tip. Further, the tip 404 can be configured to be a straight-fire fiber, a side-fire fiber, or both a straight and side firing fiber.

Optical fiber 400 includes a shaft 406 connecting the coupler 402 and tip 404. The shaft can comprise an exterior jacket 408 and a fiber core (see FIG. 2A, FIG. 2B, FIG. 3A, and FIG. 3B) disposed within the jacket 408. In some embodiments, shaft 406 can be like the optical fiber 200 or optical fiber 300 described above. For example, in some embodiments jacket 408 can be like jacket 206 and cladding 204 and fiber core 202 can be disposed within the jacket 408 as detailed above. In other embodiments, jacket 408 can be like jacket 306 and cladding 304 and fiber core 302 can be disposed within the jacket 408 as detailed above.

As described above, the optical fibers provided herein include an outer diameter (e.g., overall D 212, overall D 312, etc.) suitable for use with a conventional endoscope working channel while having a significantly smaller than conventional fiber core diameter (e.g., core D 208, core D 308, etc.). For example, even where the fiber core diameter is as small as described above, the outer diameter of shaft 406 (e.g., corresponding to overall D 212, overall D 312, or the like) can be greater than or equal to 0.660 mm, greater than or equal to 0.720 mm, greater than or equal to 0.860 mm, between 0.720 mm and 0.860 mm, or between 0.700 mm and 1.2 mm.

As another example, medical optical fibers (e.g., 200, 300, 400, or the like) can be provided as disclosed herein where the ratio of the diameter of the fiber core (e.g., core D 208, core D 308, etc.) over the overall diameter of the fiber (e.g., overall D 212, overall D 312, etc.) (e.g., D208/D212 or D308/D312, or the like) is less than or equal to 0.75, less than or equal to 0.6, less than or equal to 0.5, less than or equal to 0.4, less than or equal to 0.3, less than or equal to 0.2, less than or equal to 0.1, between 0.1 and 0.4, between 0.1 and 0.5, between 0.1 and 0.6, or between 0.1 and 0.75.

Said differently, medical optical fibers (e.g., 200, 300, 400, or the like) can be provided as disclosed herein where the ratio of the overall diameter of the fiber (e.g., overall D 212, overall D 312, etc.) over the diameter of the fiber core (e.g., core D 208, core D 308, etc.) (e.g., D212/D208 or D312/D308, or the like) is greater than or equal to 3, greater than or equal to 4, greater than or equal to 5, greater than or equal to 6, or between 3 and 6.

The fiber core of optical fiber 400 can extend from the most proximal end to the most distal end. That is, the fiber core can extend from the coupler 402 to the tip 404. In such a manner, optical fiber 400 can be configured to receive laser energy (e.g., laser energy 108) from a laser console (e.g., laser console 102) and convey the laser energy to a target. This is further described in FIG. 5.

FIG. 5 illustrates an endoscope 500, which can be a fURS or other type of endoscope (e.g., a hysteroscope, a bronchoscope, a cystoscope, etc.) Endoscope 500 can be used with optical fibers as outlined herein. For example, endoscope 500 is depicted used with optical fiber 400 for purposes of clarity of presentation. However, endoscope 500 could be used with optical fiber 106, optical fiber 200, optical fiber 300, or the like.

Endoscope 500 includes a handle 502 coupled with an elongate shaft 504 and console coupling conduit 506. The elongate shaft 504 can be inserted into a patient and includes one or more working channels extending along the longitudinal length of the elongate shaft 504 and terminating in one or more apertures 510 in the distal end 508 of the elongate shaft 504. In some embodiments the ID of the working channel and aperture 510 is 3.6F. The distal end 508 is depicted in enlarged view in FIG. 5. Endoscope 500 can further include a camera circuitry and can optionally include one or more lights, sensors, or other components arranged on or around the distal end 508.

The console coupling conduit 506 can include conductors configured to convey power between a console (e.g., endoscope viewing console, or the like) and the endoscope 500 and to convey signals (e.g., imaging signals, sensor signals, etc.) from the endoscope 500 to the endoscope viewing console.

The handle 502 can include an actuator 514 for steering the distal end 508 of the elongate shaft 504. Further, handle 502 can include port 516 to connect to the working channel or other internal lumens of the endoscope 500. For example, the port 516 can couple to the aperture 510 via an internal lumen through the handle 502 and the elongate shaft 504 such that various medical devices may be inserted through the elongate shaft 504. For example, FIG. 5 illustrates shaft 406 of optical fiber 400 inserted into the port 516 and the tip 404 extended out from the aperture 510. In such a manner, laser energy can be conveyed from the tip 404 to a target as described above.

In some embodiments, the port 516 can be a “T” connector. In some embodiments, the port 516 can include any of a variety of connection mechanisms (e.g., Luer lock, or the like). Port 516 can include a valve 518 configured to form a seal, by itself or in combination with another element passing through the valve. For example, valve 518 can be configured to form a seal when shaft 406 of optical fiber 400 is inserted through the port 516.

Accordingly, the present disclosure provides optical fibers (e.g., optical fiber 106, optical fiber 200, optical fiber 300, optical fiber 400, or the like) configured to be used with conventional endoscopes having conventional working channels (e.g., endoscope 500, or the like) having conventionally sized working channels where the optical fibers have a fiber core with a diameter substantially smaller than conventional.

As such, the present disclosure provides advantages over the prior art in that optical fibers that boost or increase the power density of delivered laser energy yet are not prone to disadvantages of conventionally thin fibers is provided. For example, thin fibers (e.g., conventional fibers with small fiber core diameters) vibrate and do not remain in a constant position due both to the low rigidity of the fiber and the size mismatch between the fiber OD and the endoscope working channel ID. Additionally, manipulating the fiber (e.g., moving it back and forward, introducing it into an endoscope, etc.) is significantly more difficult to perform with very thin fibers, especially when wearing wet surgical gloves as is often the case for practitioners performing lithotripsy procedures.

In the discussion, unless otherwise stated, adjectives such as “substantially” and “about” modifying a condition or relationship characteristic of a feature or features of an embodiment of the disclosure, are understood to mean that the condition or characteristic is defined to within tolerances that are acceptable for operation of the embodiment for an application for which it is intended. Unless otherwise indicated, the word “or” in the description and claims is the inclusive “or” rather than the exclusive or, and indicates at least one of, or any combination of items it conjoins.

While the presented concepts have been described with respect to a limited number of embodiments, it will be appreciated that many variations, modifications, and other applications of the disclosure can be implemented without departing from the scope of the appended claims.

What is claimed is:

1. A medical optical fiber, comprising:
a proximal connector, a distal tip, and an elongate shaft extending between the proximal connector and the distal tip,
the elongate shaft comprising:
a fiber core;
a cladding surrounding the fiber core; and
a jacket surrounding the cladding,
wherein a ratio of the diameter of the fiber core over the diameter of the elongate shaft is less than or equal to 0.75.

2. The medical optical fiber of claim 1, wherein the ratio of the diameter of the fiber core over the diameter of the elongate shaft less than or equal to 0.5.

3. The medical optical fiber of claim 1, wherein the ratio of a diameter of the cladding over the diameter of the fiber core is greater than or equal to 1.5.

4. The medical optical fiber of claim 1, wherein the ratio of a diameter of the cladding over the diameter of the fiber core is greater than or equal to 2.5

5. The medical optical fiber of claim 1, wherein the ratio of a diameter of the cladding over the diameter of the fiber core is greater than or equal to 1.05 and less than or equal to 1.25.

6. The medical optical fiber of claim 5, wherein the ratio the diameter of the jacket over a diameter of the cladding is greater than or equal is greater than or equal to 1.5.

7. The medical optical fiber of claim 5, wherein the ratio the diameter of the jacket over a diameter of the cladding is greater than or equal is greater than or equal to 2.5.

8. The medical optical fiber of claim 1, wherein the proximal connector is configured to mechanically couple the medical optical fiber to a laser console and to optically couple the fiber core to the laser console.

9. The medical optical fiber of claim 8, wherein the proximal connector comprises radio frequency identification (RFID) circuitry configured to authenticate the medical optical fiber to the laser console.

10. A medical laser system, comprising:
a laser console comprising a laser source and a controller, the controller configured to identify laser pulse parameters and to cause the laser source to generate laser energy having the laser pulse parameters; and
a medical optical fiber, comprising:
a proximal connector, a distal tip, and an elongate shaft extending between the proximal connector and the distal tip,
the elongate shaft comprising:
a fiber core;
a cladding surrounding the fiber core; and
a jacket surrounding the cladding,
wherein a ratio of the diameter of the fiber core over the diameter of the elongate shaft is less than or equal to 0.75.

11. The medical laser system of claim 10, wherein the laser pulse parameters comprise pulse power of 1.5 kilo-Watts (kW) and wherein the diameter of the fiber core is such that the medical optical fiber, responsive to receiving laser energy from the laser console having 1.5 kW of pulse power delivers laser energy from a distal end of the elongate shaft having a power density of 3.6 Mega-Watts (MW) per centimeter (cm) squared (MW/cm2).

12. The medical laser system of claim 10, wherein the laser pulse parameters comprise pulse power of less than or equal to 2.5 kilo-Watts (KW) and wherein the diameter of the fiber core is such that the medical optical fiber, responsive to receiving laser energy from the laser console having less than or equal to 2.5 kW of pulse power delivers laser energy from a distal end of the elongate shaft having a power density of greater than or equal to 3 Mega-Watts (MW) per centimeter (cm) squared (MW/cm2).

13. The medical laser system of claim 10, comprising an endoscope having a working channel configured to receive the medical optical fiber, wherein the working channel comprises an inner diameter (ID) of between 3.2 and 3.8 French (F).

14. The medical laser system of claim 10, wherein the ratio of a diameter of the cladding over the diameter of the fiber core is greater than or equal to 1.5.

15. The medical laser system of claim 10, wherein the ratio the diameter of the jacket over a diameter of the cladding is greater than or equal is greater than or equal to 1.5.

16. A medical optical fiber, comprising:
a proximal connector, a distal tip, and an elongate shaft extending between the proximal connector and the distal tip,
the elongate shaft comprising:
a fiber core;
a cladding surrounding the fiber core; and
a jacket surrounding the cladding,
wherein a diameter of the fiber core is greater than or equal to 100 millimeters (mm) and less than or equal to 200 mm, and
wherein a diameter of the elongate shaft is greater than or equal to three (3) times the diameter of the fiber core.

17. The medical optical fiber of claim 16, wherein the ratio of a diameter of the cladding over the diameter of the fiber core is greater than or equal to 2.5

18. The medical optical fiber of claim 16, wherein the ratio a diameter of the jacket over a diameter of the cladding is greater than or equal is greater than or equal to 2.5.

19. The medical optical fiber of claim 16, wherein the proximal connector is configured to mechanically couple the medical optical fiber to a laser console and to optically couple the fiber core to the laser console.

20. The medical optical fiber of claim 16, wherein the proximal connector comprises radio frequency identification (RFID) circuitry configured to authenticate the medical optical fiber to the laser console.
